(19) United States
(12) Patent Application Publication
(10) Pub. No.: US 2024/0347136 A1
(43) Pub. Date: Oct. 17, 2024

(54) Feature Screening Method and Apparatus, Storage Medium and Electronic Device

(71) Applicants: JIANGSU QLIFE MEDICAL TECHNOLOGY GROUP CO., LTD., Nanjing, Jiangsu (CN); NANJING QLIFE MEDICAL TECHNOLOGY CO., LTD., Nanjing, Jiangsu (CN)

(72) Inventors: Xiaoliang CHENG, Nanjing, Jiangsu (CN); Lei ZHANG, Nanjing, Jiangsu (CN); Yue ZHOU, Nanjing, Jiangsu (CN); Wei ZHANG, Nanjing, Jiangsu (CN); Kejia ZHENG, Nanjing, Jiangsu (CN)

(21) Appl. No.: 18/036,947

(22) PCT Filed: Aug. 17, 2022
(86) PCT No.: PCT/CN2022/113011

§ 371 Date: May 15, 2023

(30) Foreign Application Priority Data
Jun. 2, 2022 (CN) 202210624370.7

(51) Int. Cl. G16B 45/00; G16B 20/40; G16B 40/10; G16B 50/20

(52) U.S. Cl. CPC G16B 45/00; G16B 20/40; G16B 40/10; G16B 50/20

(57) ABSTRACT

Disclosed are a feature screening method and apparatus, a storage medium, and an electronic device. The method includes: determining feature validation subsets based on data features in sample data; partitioning the sample data into individual sample groups corresponding to different individuals based on an individual to which the sample data belongs, and performing cross-validation partitioning based on the individual sample groups, to determine a training dataset and a validation dataset obtained through partitioning; training a machine learning model of a processing target based on the training and validation datasets corresponding to each feature validation subset; and determining a target data feature group corresponding to the processing target based on training process data of each model. Thereout, cross-validation partitioning makes sample data of one individual not being partitioned into training and validation datasets meanwhile, avoiding impact of individual sample data on performance of the model and improving accuracy of feature screening.

This application claims the priority to Chinese Patent Application No. 202210624370.7, filed with the China National Intellectual Property Administration on Jun. 2, 2022 and entitled “FEATURE SCREENING METHOD AND APPARATUS, STORAGE MEDIUM, AND ELECTRONIC DEVICE”, which is incorporated herein by reference in its entirety.

### FIELD OF THE INVENTION

The present disclosure relates to the technical field of data processing, and in particular, to a feature screening method and apparatus, a storage medium, and an electronic device.

### BACKGROUND OF THE INVENTION

Currently, the mass spectrometry technology is booming and is widely applied to clinical detection projects in multiple fields, including endocrine, cardiovascular, tumor, and drug therapy. The mass spectrometry technology is an essential tool for achieving accurate diagnosis and accurate medical treatment. Big data about a plurality of omics such as proteomics, metabolomics, and lipomics of a clinical sample can be obtained based on the mass spectrometry technology. Accordingly, how to reasonably and effectively analyze multi-secretomics data brought by the mass spectrometry technology is one of key points and hotspots of research.

During implementation of the present disclosure, it is found that there are at least the following technical problems in the prior art: too many data features make it difficult to determine an effective marker from a large amount of data features. At the same time, one individual may generate a plurality pieces of sample data, and a difference between individuals leads to certain deviations in screening of the data features.

### SUMMARY OF THE INVENTION

The present disclosure provides a feature screening method and apparatus, a storage medium, and an electronic device, so as to improve the accuracy of feature screening.

According to an aspect of the present disclosure, a feature screening method is provided, including:
determining a plurality of feature validation subsets based on data features in sample data;
performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
training a machine learning model of a processing target based on the training dataset and the validation dataset corresponding to each feature validation subset; and
determining a target data feature group corresponding to the processing target based on training process data of each machine learning model.

According to another aspect of the present disclosure, a feature screening apparatus is provided, including:
a feature validation subset determining module, configured to determine a plurality of feature validation subsets based on data features in sample data;
a dataset partitioning module, configured to perform, based on an individual to which the sample data belongs, individual group partitioning on the sample data to obtain individual sample groups corresponding to different individuals, and perform cross-validation partitioning based on a plurality of individual sample groups, to determine a training dataset and a validation dataset that are obtained through partitioning;
a model training module, configured to train a machine learning model of a processing target based on the training dataset and the validation dataset corresponding to each feature validation subset; and
a target data feature group determining module, configured to determine a target data feature group corresponding to the processing target based on training process data of each machine learning model.

According to another aspect of the present disclosure, an electronic device is provided, where the electronic device includes:
at least one processor; and
a memory in a communication connection with the at least one processor,
wherein the memory stores a computer program that can be executed by the at least one processor, and the computer program is executed by the at least one processor to enable the at least one processor to implement the feature screening method according to any one of embodiments of the present disclosure.

According to another aspect of the present disclosure, a computer readable storage medium is provided, wherein the computer readable storage medium stores computer instructions, and the computer instructions are used to enable a processor to implement the feature screening method according to any one of embodiments of the present disclosure.

In the technical solutions provided in the embodiments, by validating a plurality of data features in the sample data in a form of the feature validation subset and by means of the machine learning model, wrapped screening of the data features is implemented to obtain the target data feature group for processing a target prediction. Further, for the sample data used for training the machine learning model, individual partitioning is performed to partition the sample data of a same individual into a same individual sample group, and cross-validation partitioning is performed based on the individual sample groups, to prevent the sample data of a same individual from being partitioned into the training dataset and the validation dataset at the same time, thereby avoiding impact of individual sample data on performance of the machine learning model, and further improving accuracy of feature screening.

It should be understood that the content described in this section is not intended to identify key or important features of the embodiments of the present disclosure, and also is not intended to limit the scope of the present disclosure. Other features of the present disclosure would be easily understood according to the following description.

### BRIEF DESCRIPTION OF THE DRAWINGS

To more clearly describe the technical solutions in the embodiments of the present disclosure, the accompanying drawings required in the description of the embodiments are briefly illustrated below. Apparently, the accompanying drawings in the description below are merely some embodiments of the present disclosure, and other accompanying drawings may also be obtained by one of ordinary skills in the art according to these accompanying drawings without an effective effort.

FIG. 1 is a flowchart of a feature screening method according to an embodiment of the present disclosure;

FIG. 2 is a flowchart of a feature screening method according to an embodiment of the present disclosure;

FIG. 3 is a flowchart of a feature screening method according to an embodiment of the present disclosure;

FIG. 4 is an example diagram of a data distribution map according to an embodiment of the present disclosure;

FIG. 5 is a flowchart of a feature screening method according to an embodiment of the present disclosure;

FIG. 6 is a schematic structural diagram of a feature screening apparatus according to an embodiment of the present disclosure; and

FIG. 7 is a schematic structural diagram of an electronic device according to an embodiment of the present disclosure.

### DETAILED DESCRIPTION OF THE EMBODIMENTS

To make a person skilled in the art better understand the solutions of the present disclosure, the technical solutions in the embodiments of the present disclosure are clearly and completely described below in combination with the accompanying drawings in the embodiments of the present disclosure. Obviously, the described embodiments are merely some embodiments of the present disclosure and are not all embodiments. According to the embodiments in the present disclosure, all other embodiments derived by one of ordinary skills in the art without an effective effort shall belong to the protection scope of the present disclosure.

It should be noted that the terms “first”, “second”, and the like in this specification, the claims, and the accompanying drawings of the present disclosure are intended to distinguish between similar objects, but are not necessarily intended to describe a particular sequence or a sequential order. It should be understood that data used in this way can be interchanged in appropriate cases, so that the embodiments of the present disclosure described herein can be implemented in an order other than those illustrated or described herein. In addition, the terms “include” and “has”, and any variations thereof are intended to cover non-exclusive inclusion. For example, a process, a method, a system, a product, or a device that includes a series of steps or units is not necessarily limited to those clearly listed steps or units, but may include other steps or units that are not clearly listed or are inherent to the process, the method, the product, or the device.

### Embodiment 1

FIG. 1 is a flowchart of a feature screening method according to an embodiment of the present disclosure. This embodiment can be applied to a case of screening a data feature for predicting a processing target among a large quantity of data features. The method can be implemented by a feature screening apparatus. The feature screening apparatus can be implemented in a form of hardware and/or software. The feature screening apparatus can be configured in an electronic device such as a computer or a server, etc., As shown in FIG. 1, the method includes:
S110, determining a plurality of feature validation subsets based on data features in sample data;
S120, performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
S130, training a machine learning model of a processing target based on the training dataset and the validation dataset corresponding to each feature validation subset; and
S140, determining a target data feature group corresponding to the processing target based on training process data of each machine learning model.

In this embodiment, a large amount of sample data is obtained. Each group of sample data can include various types of data features. Types of data features included in different groups of sample data may be the same, and data values of the data features are different. Optionally, the sample data can include secretomics data and/or clinical data. For example, the secretomics data can be obtained through the mass spectrometry technology. The secretomics data includes, but is not limited to proteomics, metabolomics, and lipomics. The clinical data may be collected by using a data collection device, or may be historical collection data, and the clinical data includes but is not limited to blood pressure, heart rate, respiratory frequency, and the like. Each data feature in the sample data can be recorded in the following way: {(xᵢyᵢ)}ᵢ₌₁^{N}, where xᵢ represents an i^{th} sample feature vector, and N represents i=1, . . . , N sample feature vectors. A dimension of xᵢ is recorded as j=1, . . . , D, and each dimension x^{j} represents a j^{th} feature. There are D features in total. yᵢ represents a label of xᵢ, a value of yᵢ is a real number, and a feature label y is of a numerical type.

There are many types of data features in the sample data, and only some data features have an impact on the processing target. To be specific, target data features corresponding to the processing target are merely some features of the data features in the sample data, and target data features corresponding to different processing targets may be different. It should be noted that the processing target may be a partition prediction of input data on any dimension. For example, the processing target is a prediction of a hormone concentration based on different time points, or is a prediction of a pathological grading based on a certain disease. It should be noted that in the foregoing sample data, yᵢ represents a label of xᵢ on a dimension of the processing target.

In some embodiments, based on the data features in each group of sample data, a data feature set is obtained, and a plurality of feature validation subsets are randomly determined in the data feature set. A quantity of data features in the feature validation subset is random, and the quantity of the data features may be larger than 1 but less than a total quantity of the data features. In other words, the feature validation subset can include some or all the data features. Based on the determined quantity of the data features, a corresponding quantity of data features are randomly determined in the data feature set, to form the feature validation subset.

Optionally, said determining a plurality of feature validation subsets based on the data features in the sample data includes: determining a plurality of feature validation subsets in the data features in the sample data based on the quantity of the features in the feature validation subset. Optionally, the quantity of the features in the feature validation subset may be preset, and it may be set according to user requirements. For example, the quantity may be 8, 10, or 15, etc. Optionally, the quantity of the features in the feature validation subset may also be determined based on a data volume of the sample data. A maximum quantity of the features in the feature validation subset is a ratio of a quantity of samples to a preset value. The preset value may be 15. It should be noted that the preset value is not limited, and can be set according to user requirements. Correspondingly, the quantity of the features in the feature validation subset is located in

$$[ 1 , \frac{the number of samples}{15} ] ,$$

where the number of samples represents the quantity of the samples. The quantity of the feature validation subsets can be determined based on a quantity d of the features in the feature validation subset and a total quantity D of the data features in the sample data. For example, the quantity of the feature validation subsets is

$$C = \frac{D !}{d ! ( D - d ) !} .$$

Wrapped feature selection is performed on the plurality of data features in the feature validation subset, so that the target data feature of the processing target, that is, a combination of important markers for predicting the label y, is obtained through screening. Specifically, by validating the machine learning model for each feature validation subset, accuracy of the feature validation subset is reversely validated through a training result of the machine learning model, thereby obtaining the target data feature group corresponding to the processing target.

On the basis of the foregoing embodiments, before training each machine learning model based on the sample data, cross-validation partitioning is performed on the sample data to obtain the training dataset and the validation dataset. In combination with a correspondence between the sample data and the individual, cross-validation partitioning is performed on the sample data, thereby preventing the sample data from a same individual from being partitioned into the training dataset and the validation dataset at the same time, which affects actual performance of the machine learning model. For example, N pieces of sample data may come from M individuals, where M≤N. A quantity M of the individuals can be equal to a quantity N of the samples, and the sample data may also be M samples at different stages. If it is satisfied that M=N, it indicates that an individual sₘ and a sample xᵢ have a unique one-to-one correspondence. In other words, each individual uniquely corresponds to one sample, where m and i represent the same sample. In this case, it is satisfied that sₘ=sᵢ, and data satisfies that {(sₘxᵢyᵢ)}={(sᵢxᵢyᵢ)} If it is satisfied that M<N, it indicates that sₘ and xᵢ are in a one-to-many relationship. This type of data is a collection of a plurality of samples for one individual. For example, an m^{th} individual satisfies that {xᵢ₌₁, xᵢ₌₂, . . . , xᵢ₌ᵢ}, which indicates that there are l samples x from a same individual in the samples.

Optionally, performing, based on the individual to which the sample data belongs, individual group partitioning on the sample data to obtain the individual sample groups corresponding to different individuals, and performing cross-validation partitioning based on a plurality of individual sample groups, to determine the training dataset and the validation dataset that are obtained through partitioning includes: partitioning at least one group of sample data of a same individual to a same individual group, to obtain individual sample groups corresponding to different individuals; and performing cross-validation partitioning on the plurality of individual sample groups based on at least one preset cross validation rule, to determine the training dataset and the validation dataset that are obtained through partitioning.

Each piece of sample data can carry identification information of the individual to which the sample data belongs. Various sample data can be partitioned based on the identification information of the individuals to which the sample data belongs. To be specific, sample data carrying same identification information is partitioned into a same individual group, thereby obtaining various individual sample groups. It is satisfied that m=1, . . . , M. Corresponding sample set of each individual are found in sequence, and are record as sₘ={xᵢ₌₁, xᵢ₌₂, . . . , xᵢ₌ₗ}. Each individual sample group is used as a unit data group for the cross-validation partitioning, and the cross-validation partitioning is performed to obtain the training dataset and the validation dataset.

In this embodiment, an implementation of the cross-validation partitioning is not limited, provided that the cross-validation partitioning can be performed on any individual sample group. For example, a repeated K-fold cross-validation manner, a Leave-One-Out cross-validation manner, and a Leave-P-Out cross-validation manner can be used. The individual sample group can be performed with cross-validation partitioning based on any one of the foregoing cross-validation partitioning manners. A configuration parameter K in the repeated K-fold cross-validation (Repeated K-fold) manner is an integer larger than or equal to 2; and a quantity of repetitions, that is, Repeated, is an integer larger than or equal to 1. For example, it is default that K=10 and Repeated=10. For example, when a quantity of individual data groups satisfies M<10, it is default that K=3 and Repeated=5. For the leave-one-out cross-validation (LeaveOneOut) manner, there is no need to set a parameter, and it may be default that the parameter K=M and Repeated=1. For the Leave-P-Out cross-validation (LeavePOut) manner, a value range of P is 1≤P≤M. In this case, only a parameter P exits, indicating that P individual datasets are used as a test dataset, and M-P individuals are used as a training dataset.

It should be noted that when the quantity of the individual data groups is less than a quantity threshold such as 100, cross-validation partitioning can be performed on the individual sample groups by using the Leave-One-Out cross-validation manner and the Leave-P-Out cross-validation manner, to ensure stability of a running result of the training of the machine learning model.

For example, for the repeated K-fold cross-validation manner or the Leave-One-Out cross-validation manner, cross-validation partitioning is performed on the sample data: M individual sample groups are randomly divided into K subset folds, which are recorded as {F₁, F₂, . . . , Fₖ}. For each subset, each subset is used as a test dataset, and K−1 subsets are used as training datasets, which are recorded as CV(r=1). The quantity of repetitions is Repeated. A data partitioning result is recorded as CVᵣ₌₁^{repeated}, where

$$C {V}_{r = 1} = [ \begin{matrix}testset ( {F}_{11} ) , trainset ( {F}_{12} , … , {F}_{1 K} ) \\ testset ( {F}_{12} ) , trainset ( {F}_{11} , {F}_{13} , … , {F}_{1 K} ) \\ … , … \\ testset ( {F}_{1 k} ) , trainset ( {F}_{11} , {F}_{12} , … , {F}_{1 ( K - 1 )} )\end{matrix} ] ;$$

$$and C {V}_{r = 1}^{repeated} = [ \begin{matrix}testset ( {F}_{repeated 1} ) , trainset ( {F}_{repeated 2} , … , {F}_{repeated K} ) \\ testset ( {F}_{repeated 2} ) , trainset ( {F}_{repeated 1} , {F}_{repeated 3} , … , {F}_{repeatedK} ) \\ … , … \\ testset ( {F}_{repeatedk} ) , trainset ( {F}_{repeated 1} , {F}_{repeated} , … , {F}_{repeated ( K - 1 )} )\end{matrix} ] ,$$

where testset(*) represents the test dataset, and trainset(*) represents the training dataset.

For the Leave-P-Out cross-validation manner, the cross-validation partitioning is performed on the sample data: P individual data groups are taken from M individual data groups for combination, where a quantity of combinations is

$$C ( M , P ) = \frac{M !}{P ! ( M - P ) !} .$$

For each of the C(M,P) combinations: P individual data groups are used as test datasets {Sₗ, . . . , Sₚ}, and M-P individuals are used as training datasets {S₍ₚ₊₁₎, . . . , S_{M}}. The data partitioning result is recorded as CV_{C=1}^{C(M,P)}=[testset(S_{C1}, . . . , S_{CP}), trainset(S_{C(P+1)}, . . . , S_{C(CM)})].

On the basis of the foregoing embodiments, the machine learning model is performed with processing target-based training based on the training dataset and the validation dataset that are obtained through cross-validation partitioning. Because types and quantities of the data features included in different feature validation subsets are different, based on the type of the data feature in each feature validation subset, corresponding data items are screened out from various training datasets and various validation datasets that are described above, to form the training dataset and the validation dataset corresponding to the feature validation subset. The machine learning model is trained based on the training dataset and the validation dataset corresponding to the feature validation subset, to obtain the corresponding machine learning model of the feature validation subset based on the processing target.

In this embodiment, the machine learning model may be a logistic regression model. For example, the machine learning model includes, but is not limited to a simple linear regression model, a ridge regression model, a lasso regression model, an elastic network regression model, a Bayesian regression model, a k-nearest neighbor regression model, a support vector machine regression model, and a random forest regression model. For each feature validation subset, one or more of the foregoing logistic regression models can be adopted for model training, and grid search can be used to optimize a model parameter during the training process.

In a process of training the machine learning model of the processing target based on each feature validation subset, a plurality of machine learning models are obtained based on a same training manner. The same training manner includes, but is not limited to a same quantity of samples, a same loss function, a same learning rate, and a same quantity of iterations. For the machine learning model that has completed training, optionally, a training result of the machine learning model can include but is not limited to a first parameter used to indicate the degree of training completion and a second parameter used to indicate model accuracy. Optionally, the training result of the machine learning model can include but is not limited to the evaluation information of prediction of the model. An optimal machine learning model is screened out by using one or more of the forgoing parameters or the prediction and evaluation information. Correspondingly, the feature validation subset corresponding to the optimal machine learning model may be determined as the target data feature group. Optionally, the training result of the machine learning model may be a prediction result of the sample data. An evaluation parameter of the machine learning model, such as a prediction error, may be determined through the label in the sample data and the prediction result. The evaluation parameter of the machine learning model can be used to sequence various machine learning models, or to screen out the optimal machine learning model, thereby determining the target data feature group corresponding to the processing target.

In some embodiments, determining the target data feature group corresponding to the processing target based on the training process data of each machine learning model includes: for any machine learning model, respectively determining a training indicator and a test indicator based on training data and validation data in the training process data of the machine learning model; sequencing and screening various machine learning models based on the training indicator and the test indicator of each machine learning model; and determining a feature validation subset corresponding to a screened machine learning model as the target data feature group corresponding to the processing target.

The training data is a prediction result obtained by training the machine learning model based on the sample data in the training dataset, and the validation data is a prediction result obtained by training the machine learning model based on the sample data in the validation dataset. There is at least one indicator type for each of the training indicator and the test indicator, and the indicator type of the training indicator and the test indicator are the same. For example, the training indicator and the test indicator respectively include a root-mean-square error RMSE and a goodness of fit R².

For example, the root-mean-square error RMSE can be calculated by using the following formula:

$$R M S E = \sqrt{\frac{1}{N} {\sum }_{i = 1}^{N} {( {y}_{i} - {\hat{y}}_{i} )}^{2}} .$$

The goodness of fit R² can be calculated by using the following formula:

$${R}^{2} = 1 - \frac{{\sum }_{i = 1}^{N} {( {y}_{i} - {\hat{y}}_{i} )}^{2}}{{\sum }_{i = 1}^{N} {( {y}_{i} - {\overline{y}}_{i} )}^{2}} ,$$

where ŷᵢ represents a predicted value; yᵢ represents a real value, i.e., a label value in the sample data; and y̅ᵢ represents an average value of the real value.

Based on various machine learning models obtained through the processing target-based training, the training indicators and the test indicators of the machine learning models, and a correspondence between the feature validation subsets corresponding to the machine learning models, the target data feature group corresponding to the processing target is determined by using the training indicator and the testing indicator of the machine learning model. In other words, the feature validation subset corresponding to the machine learning model whose training indicator and test indicator meet a screening condition is determined as the target data feature group corresponding to the processing target.

Optionally, the evaluation parameter is determined based on the root-mean-square error RMSE in the training indicator and the root-mean-square error RMSE in the test indicator. In other words, the evaluation parameter is an absolute value of a difference between the root-mean-square error RMSE in the training indicator and the root-mean-square error RMSE in the test indicator. The evaluation parameter is negatively correlated with performance stability of the machine learning model. A smaller evaluation parameter indicates that performance of a training dataset model is closer to that of a test dataset model. In other words, performance of the machine learning model is more stable. In some embodiments, the feature validation subset corresponding to the machine learning model with an evaluation parameter less than a first preset value can be determined as the target data feature group corresponding to the processing target.

The goodness of fit R² in the test indicator is positively correlated with performance of the machine learning model. A larger goodness of fit R² in the test indicator indicates better performance of the machine learning model. In some embodiments, the feature validation subset corresponding to the machine learning model with a goodness of fit R² in the test indicator larger than a second preset value can be determined as the target data feature group corresponding to the processing target.

In some embodiments, the performance of the machine learning model can be jointly evaluated based on the evaluation parameter and the goodness of fit R² in the test indicator. For example, weighted processing is performed based on the evaluation parameter, the goodness of fit R² in the test indicator, and corresponding weights, to obtain a performance evaluation value of the machine learning model. The machine learning models are sequenced based on the performance evaluation value. The feature validation subset corresponding to the machine learning model whose performance evaluation value meets performance requirements is determined as the target data feature group corresponding to the processing target.

In the technical solutions provided in this embodiment, by validating a plurality of data features in the sample data in a form of the feature validation subset and by means of the machine learning model, wrapped screening of the data features is implemented to obtain the target data feature group for predicting a target processing. Further, for the sample data used for training the machine learning model, individual partitioning is performed to partition the sample data of a same individual into a same individual sample group, and cross-validation partitioning is performed based on the individual sample groups, so that the sample data of a same individual is prevented from being partitioned into the training dataset and the validation dataset at the same time, thereby avoiding impact of individual sample data on performance of the machine learning model, and further improving accuracy of feature screening.

### Embodiment 2

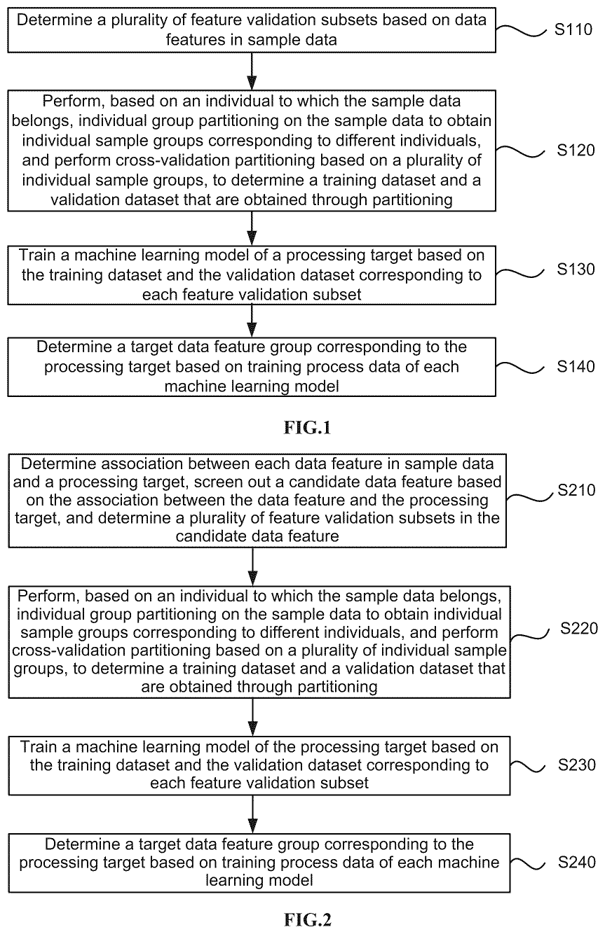

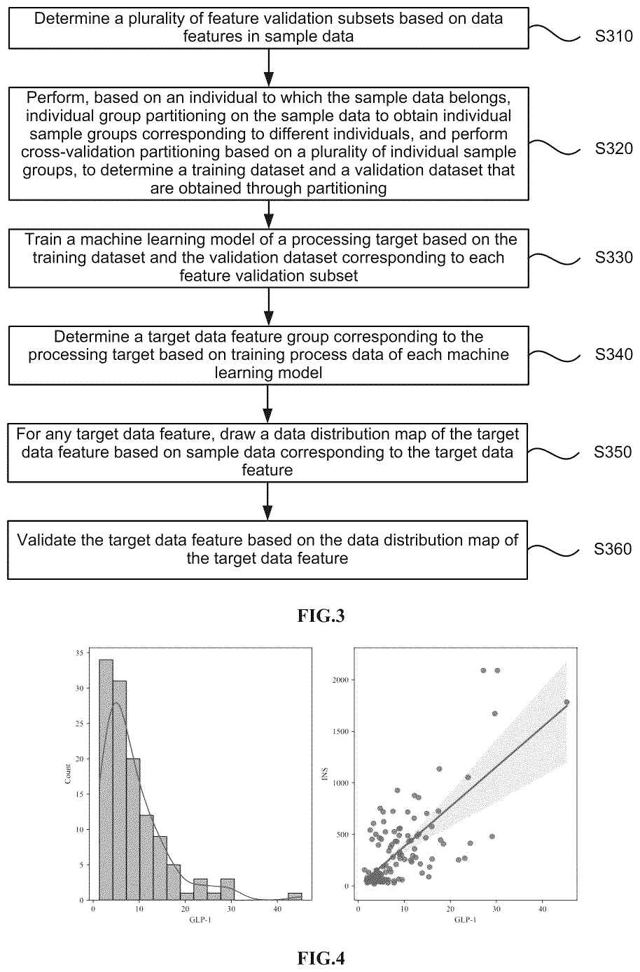

FIG. 2 is a flowchart of a feature screening method according to an embodiment of the present disclosure. Optimization is performed based on the foregoing embodiments. Optionally, before a plurality of feature validation subsets are determined based on the data features in the sample data, the method further includes: determining association between each data feature in the sample data and the processing target, and screening out a candidate data feature based on the association between the data feature and the processing target; and correspondingly, said determining a plurality of feature validation subsets based on the data features in the sample data includes: determining a plurality of feature validation subsets in the candidate data feature.

As shown in FIG. 2, the method includes:
S210, determining association between each data feature in sample data and a processing target, screening out a candidate data feature based on the association between the data feature and the processing target, and determining a plurality of feature validation subsets in the candidate data feature;
S220, performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
S230, training a machine learning model of the processing target based on the training dataset and the validation dataset corresponding to each feature validation subset; and
S240, determining a target data feature group corresponding to the processing target based on training process data of each machine learning model.

A large quantity of data features in the sample data correspondingly results in problems that there is a large quantity of feature validation subsets, and the screening process of the target data feature group is computationally expensive and time-consuming. In this embodiment, before determining a plurality of feature validation subsets, preliminary screening is performed on the data features in the sample data, and a data feature that is not related to or has a weak association with the processing target is removed, so as to obtain a candidate data feature that is related to or has a strong association with the processing target. Compared to all data features in the sample data, a quantity of the candidate data features is reduced, thereby reducing computational amount of the screening process and improving screening efficiency at the same time.

In this embodiment, the candidate data feature is determined based on the association between each data feature and the processing target. The association between the data feature and the processing target can be characterized by a numerical value, and whether the data feature is associated with the processing target and strength of the association are determined by using a comparison result of the numerical value and a threshold.

The association between the data feature and the processing target can be determined through at least one association determining rule, to calculate the association between the data feature and the processing target from different dimensions, thereby improving accuracy of the determined candidate data feature. In some embodiments, screening can be performed on the data features in the sample data for a plurality of times based on the association determined based on a plurality of association determining rules. For example, a first association between the data feature and the processing target is determined based on a first association determining rule. Based on the first association corresponding to each data feature, the data feature that is not related to or has a weak association with the processing target is removed, to obtain a first candidate data feature. For the first candidate data feature, a second association between the data feature and the processing target is determined based on a second association determining rule. Based on the second association corresponding to each first candidate data feature, the data feature that is not related to or has a weak association with the processing target is removed, to obtain a second candidate data feature. The others are deduced by analogy, until a final candidate data feature is obtained.

Optionally, the association determining rules include, but are not limited to a univariate linear regression method, a mutual information method, and a lasso regression method. In some embodiments, association calculation is respectively performed on the data features in the sample data based on the foregoing association determining rules in sequence, and the candidate data feature is screened out.

For the univariate linear regression method, a linear equation wx+b can be constructed for the data feature and the processing target, where w represents a slope, and b represents an intercept. An absolute value of the slope is positively correlated with the association, and a first association P value between the data feature and the processing target can be calculated through the slope. A smaller first association between the data feature and the processing target indicates a strong association between the data feature and the processing target, while a larger first association between the data feature and the processing target indicates a weak association between the data feature and the processing target. Correspondingly, if the first association of the data feature is less than a preset association threshold, the feature data is set as the candidate data feature. In other words, feature data with a first association P value larger than or equal to the preset association threshold is removed. The preset association threshold may be 0.1 or 0.05, which can be determined based on set screening accuracy.

For the mutual information method, a second association MI value between the data feature and a label y (that is, the processing target) can be calculated according to the following formula:

$$M I ( {x}^{i} , y ) = log \frac{p ( {x}^{i} , y )}{p ( {x}^{i} ) p ( y )} ,$$

where p represents a probability value. If MI (x^{j}, y) between two variables is 0, it indicates that there is no association between a x^{jth} data feature and the processing target J. Correspondingly, if the second association of the data feature is not zero, the feature data is set as the candidate data feature. In other words, a data feature with a second association MI value of zero is removed.

In some embodiments, the feature data with a first association P value larger than or equal to the preset association threshold and the data feature with a second association MI value of zero can be removed from all the data features in the sample data, to obtain the first candidate data feature, which may be, for example, recorded as D_{filter1}.

On the basis of the foregoing embodiments, the obtained candidate data feature D_{filter1} is further screened by using the lasso regression method. A regression model is constructed for each candidate data feature D_{filter1} that has been screened out, for example, Σᵢ₌₁^{N}(yᵢ−Σⱼxᵢⱼβⱼ)²+λΣⱼ₌₁^{Dfilter1}|βⱼ|, where λ represents a penalty factor and βᵢ represents a coefficient value. If a x^{(j)th} feature in the model is not associated with y, correspondingly, βⱼ=0. Important features with βᵢ!=0 are screened out from D_{filter1} data features. In other words, a data feature with βᵢ=0 is removed. The final candidate data feature obtained is recorded as j−1, . . . , D_{filter2}. To be specific, there are D_{filter2} data features in total.

Optionally, the screened candidate data features can be sequenced based on an absolute value of βⱼ. A larger absolute value of βⱼ indicates a larger association between the data feature and the processing target.

A plurality of feature validation subsets are determined based on the candidate data features, to further screen out a target data feature group of the processing target by means of machine learning.

According to the technical solutions of this embodiment, through the association between each data feature and the processing target, preliminary screening is performed on all data features to remove some non-associated or weakly associated technical features, thereby reducing a quantity of features for screening in a machine learning process. Further, screening is performed based on the candidate technical feature to obtain the target data feature group corresponding to the processing target, thereby reducing the quantity of data features to selectively screen the candidate data feature, and reducing interference of invalid data features and computational and time costs of screening.

### Embodiment 3

FIG. 3 is a flowchart of a feature screening method according to an embodiment of the present disclosure. Optimization is performed based on the foregoing embodiments. Optionally, after the target data feature group is determined, the method further includes: for any target data feature, drawing a data distribution map of the target data feature based on sample data corresponding to the target data feature; and validating the target data feature based on the data distribution map of the target data feature. As shown in FIG. 3, the method includes:
S310, determining a plurality of feature validation subsets based on data features in sample data;
S320, performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
S330, training a machine learning model of a processing target based on the training dataset and the validation dataset corresponding to each feature validation subset;
S340, determining a target data feature group corresponding to the processing target based on training process data of each machine learning model;
S350, for any target data feature, drawing a data distribution map of the target data feature based on sample data corresponding to the target data feature; and
S360, validating the target data feature based on the data distribution map of the target data feature.

In analysis of massive data features, distribution of an individual data feature in a dataset would be easily ignored. Although a screened target data feature group can construct a model with good performance, if distribution of the data feature does not conform to clinical manifestations, deviations would be easily introduced in the process of subsequent research or application of the model, which affects the performance of the model.

To avoid the foregoing problems existing in the data feature in the screened target data feature group, data distribution validation is performed on the screened target data feature, to ensure that the target data feature for predicting the processing target meets data distribution requirements. In this embodiment, the data distribution map is drawn for each target data feature to determine whether the data distribution map meets the data distribution requirements.

Optionally, drawing the data distribution map of the target data feature based on the sample data corresponding to the target data feature includes: determining a data type of the target data feature; and drawing a data distribution map corresponding to the data type based on the sample data corresponding to the target data feature.

Data types of the target data feature can include a sub type and a numerical type, and different data types correspond to different types of data distribution maps. A data feature of the sub type has limited data content for different objects, and belongs to a fixed range of data content. For a data feature of the numerical type, data of different objects is non fixed data and can be any data within a data range, without being limited to a positive number. For example, for a target data feature of some sub type, data content of any object is any item in {1, 0}. To be specific, the data content of any object is 0 or 1, and there are no other data forms. For a target data feature of some numerical type, data content of any object can be (0, 1), and correspondingly, data content of different objects is any numerical value larger than 0 but less than 1, such as 0.5, 0.33, 0.96, or 0.5689, etc.

In this embodiment, the data type of the target data feature is determined based on a value type and a quantity of data values of data content corresponding to the target data feature. The value type may be an integer type or a decimal type. For example, the value type corresponding to the target data feature of the sub type can be an integer type, and the value type corresponding to the target data feature of the numerical type can include the integer type and the decimal type. A quantity of data values can be a quantity of non-repeated data values. The target data feature of the sub type corresponds to a limited quantity of data values. Moreover, the quantity of the data values is relatively small; for example, it is less than a quantity threshold. The target data feature of the numerical type corresponds to a relatively large quantity of the data values, or the quantity of the data values is larger than the quantity threshold.

Determining the data type of the target data feature includes: performing deduplication on data values of the target data feature to obtain deduplicated data values; when each deduplicated data value is an integer and a quantity of data values is less than or equal to a preset threshold, determining that the data type of the target data feature is a sub type; and when each deduplicated data value is not an integer or the quantity of the data values is larger than or equal to the preset threshold, determining that the data type of the target data feature is a numerical type.

The data values of the target data feature in the sample data are deduplicated to remove duplicate data values, to obtain unique data values, and obtain a unique dataset of the target data feature, which can be recorded as a set s₁={x₁⁽¹⁾, . . . , xₙ⁽¹⁾}. Statistics about a quantity of data values in this dataset and value types in each data are performed. If the data value in the set is an integer and the quantity of the data values is less than or equal to the preset threshold, it is determined that the data type of the target data feature is the sub type. Correspondingly, if the data value in the set is not an integer, or the quantity of the data values in the set is larger than the preset threshold, it is determined that the data type of the target data feature is the numerical type. The preset threshold can be 5. This is not limited and can be set according to requirements. For example, if each element of s₁ is an integer and it is satisfied that n≤S, x⁽¹⁾ is recorded as sub-type data 0₁, or otherwise is recorded as numerical-type data 1₁. A determining result is stored in a vector s=(α₁), where α is 0 or 1. The sub type is represented when α is 0, and the numerical type is represented when α is 1. For other target data features, corresponding data types are determined respectively through the foregoing determining process, to obtain a data type vector s=(α₁, α₂, . . . , α_{d}) of each target data feature in initial clinical data, where α is 0 or 1. Further, the foregoing determining process can be performed on different target data features at the same time, to improve efficiency of determining the data type.

A data distribution map type of the target data feature is determined based on the data type of the target data feature. Further, drawing the data distribution map corresponding to the data type based on the sample data corresponding to the target data feature includes: if the data type of the target data feature is the sub type, drawing, based on the sample data corresponding to the target data feature, a horizontal bar chart of the target data feature, and a box chart of the target data feature and the processing target; or if the data type of the target data feature is the numerical type, drawing, based on the sample data corresponding to the target data feature, a histogram of the target data feature, and a scatter regression plot of the target data feature and the processing target.

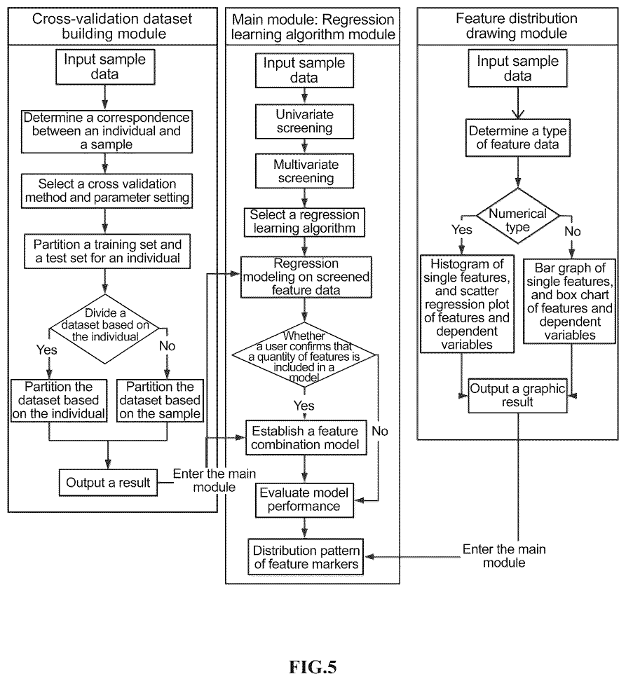

For any target data feature, a data value of the target data feature in the sample data is obtained. Based on the data distribution map type corresponding to the target data feature, the data distribution map of the target data feature is drawn by using the data value of the target data feature. For example, referring to FIG. 4, FIG. 4 is an example diagram of a data distribution map according to an embodiment of the present disclosure. The left figure in FIG. 4 is a bar chart, and the right figure is a scatter regression plot.

A respective data distribution map of the target data feature is drawn to validate whether the target data feature meets the data distribution requirements. A data distribution map of a correspondence between the target data feature and the processing target is drawn to validate whether the correspondence between the target data feature and the processing target meets the data distribution requirements.

Optionally, validating the target data feature based on the data distribution map of the target data feature includes: in response to that the data distribution map of the target data feature does not conform to a distribution rule, removing the target data feature, or removing a target data feature group to which the target data feature belongs.

In some embodiments, different data distribution map types can correspond to different distribution rules. The data distribution map of the target data feature is validated based on the distribution rule corresponding to the target data feature. In some embodiments, different target data features correspond to different distribution rules. The data distribution map of the target data feature can be validated based on a distribution rule corresponding to a type of the target data feature.

To avoid errors being introduced in subsequent analysis and application, a target data feature whose data distribution map does not conform to the distribution rule is removed. Further, due to joint action of a plurality of target data features in the target data feature group, the object that processing target is predicted is achieved. When any target data feature in the target data feature group does not conform to the distribution rule, errors are introduce in subsequent analysis and application due to the target data feature group. Therefore, the target data feature group is removed.

In the technical solutions provided in this embodiment, after the target data feature group corresponding to the processing target is screened out from a plurality of feature validation subsets by means of machine learning, data distribution validation is additionally performed on the target data feature to remove a data feature that does not conform to clinical manifestations, thereby ensuring practicality of the screened target data feature.

### Embodiment 4

On the basis of the foregoing embodiments, an embodiment of the present disclosure further provides a preferable example of a feature screening method. For example, referring to FIG. 5, FIG. 5 is a flowchart of a feature screening method according to an embodiment of the present disclosure. FIG. 5 provides a system structure for implementing the feature screening method. The system structure includes a main module (that is, a regression learning algorithm module), a cross-validation dataset building module, and a feature distribution drawing module. The main module can invoke the cross-validation dataset building module and the feature distribution drawing module. After the main module performs univariate screening on the data features in the sample data, a plurality of feature validation subsets for multivariate screening are determined. Before multivariate screening is performed through a regression learning algorithm, the cross-validation dataset building module is invoked to obtain a training dataset and a test dataset that are used for the multivariate screening. The cross-validation dataset building module is configured to partition the training dataset and the test dataset based on a correspondence between an individual and a sample by using a cross validation method. The main module implements the regression learning algorithm to obtain a multivariate screening result, that is, the target data feature group. The main module invokes the feature distribution drawing module to draw a distribution graph of features in data, thereby visually displaying a distribution status of the target data features and validating the target data feature group.

### Embodiment 5

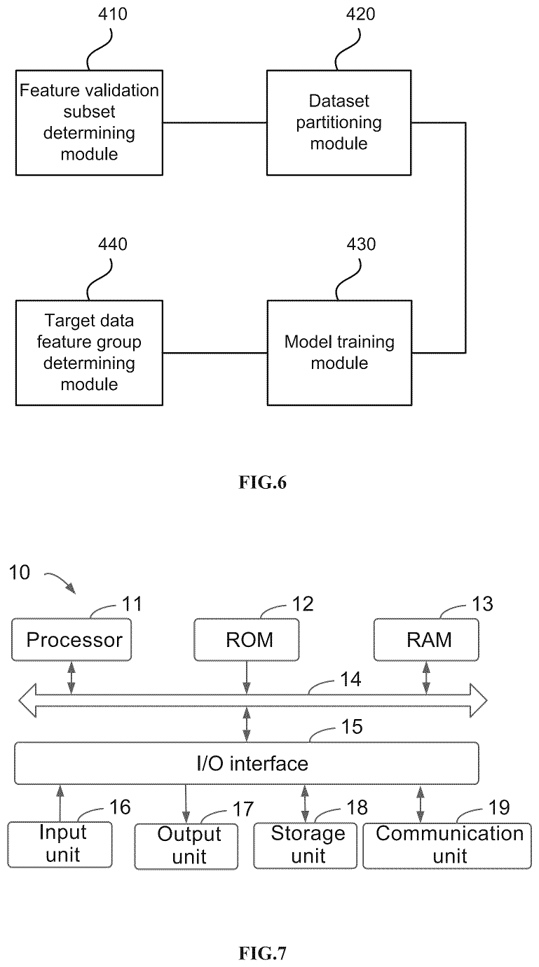

FIG. 6 is a schematic structural diagram of a feature screening apparatus according to an embodiment of the present disclosure. As shown in FIG. 6, the apparatus includes:
a feature validation subset determining module 410, configured to determine a plurality of feature validation subsets based on data features in sample data;
a dataset partitioning module 420, configured to perform, based on an individual to which the sample data belongs, individual group partitioning on the sample data to obtain individual sample groups corresponding to different individuals, and perform cross-validation partitioning based on a plurality of individual sample groups, to determine a training dataset and a validation dataset that are obtained through partitioning.
a model training module 430, configured to train a machine learning model of a processing target based on the training dataset and the validation dataset corresponding to each feature validation subset; and
a target data feature group determining module 440, configured to determine a target data feature group corresponding to the processing target based on training process data of each machine learning model.

In the technical solutions provided in this embodiment, by validating a plurality of data features in the sample data in a form of the feature validation subset and by means of the machine learning model, wrapped screening of the data features is implemented to obtain the target data feature group for predicting the processing target. Further, for the sample data used for training the machine learning model, individual partitioning is performed to partition the sample data of a same individual into a same individual sample group, and cross-validation partitioning is performed based on the individual sample groups, so that the sample data of a same individual is prevented from being partitioned into the training dataset and the validation dataset at the same time, thereby avoiding impact of individual sample data on performance of the machine learning model, and further improving accuracy of feature screening.

On the basis of the foregoing embodiment, optionally, the apparatus further includes:
a candidate data feature screening module, configured to determine, before a plurality of feature validation subsets are determined based on the data features in the sample data, association between each data feature in the sample data and the processing target, and screen out a candidate data feature based on the association between the data feature and the processing target.

Correspondingly, the feature validation subset determining module 410 is configured to determine a plurality of feature validation subsets in the candidate data feature.

On the basis of the foregoing embodiment, optionally, the feature validation subset determining module 410 is configured to determine a plurality of feature validation subsets in the data feature of the sample data or in the candidate data feature based on a quantity of features in the feature validation subsets.

On the basis of the foregoing embodiment, optionally, the dataset partitioning module 420 is configured to:
partitioned at least one group of sample data of a same individual to one individual group, to obtain individual sample groups corresponding to different individuals; and
perform cross-validation partitioning on the plurality of individual sample groups based on at least one preset cross validation rule, to determine the training dataset and the validation dataset that are obtained through partitioning.

On the basis of the foregoing embodiment, optionally, the target data feature group determining module 440 is configured to:
for any machine learning model, respectively determine a training indicator and a test indicator based on the training data and the validation data in the training process data of the machine learning model;
sequence and screen various machine learning models based on the training indicator and the test indicator of each machine learning model; and
determine a feature validation subset corresponding to a screened machine learning model as the target data feature group of the processing target.

Optionally, the training indicator and the test indicator respectively include a root-mean-square error and a goodness of fit.

On the basis of the foregoing embodiment, optionally, the apparatus further includes:
a data distribution map drawing module, configured to draw, for any target data feature, a data distribution map of the target data feature based on sample data corresponding to the target data feature; and
a feature validation module, configured to validate the target data feature based on the data distribution map of the target data feature.

Optionally, the data distribution map drawing module includes:
a data type determining unit, configured to determine a data type of the target data feature; and
a data distribution map drawing unit, configured to draw a data distribution map corresponding to the data type based on the sample data corresponding to the target data feature.

Optionally, the data type determining unit is configured to:
perform deduplication on data values of the target data feature to obtain deduplicated data values;
in response to that each deduplicated data value is an integer and a quantity of data values is less than or equal to a preset threshold, determine that the data type of the target data feature is a sub type; and in response to that each deduplicated data value is not an integer or the quantity of the data values is larger than or equal to the preset threshold, determine that the data type of the target data feature is a numerical type.

Optionally, the data distribution map drawing module is configured to:
if the data type of the target data feature is the sub type, draw, based on the sample data corresponding to the target data feature, a horizontal bar chart of the target data feature, and a box chart of the target data feature and the processing target; or
if the data type of the target data feature is the numerical type, draw, based on the sample data corresponding to the target data feature, a histogram of the target data feature, and a scatter regression plot of the target data feature and the processing target.

Optionally, the feature validation module is configured to:
in response to that the data distribution map of the target data feature does not conform to a distribution rule, remove the target data feature, or remove a target data feature group to which the target data feature belongs.

The feature screening apparatus provided in this embodiment of the present disclosure can implement the feature screening method provided in any embodiment of the present disclosure, and has corresponding functional modules and beneficial effects for implementing the method.

### Embodiment 6

FIG. 7 is a schematic structural diagram of an electronic device according to Embodiment 6 of the present disclosure. An electronic device 10 is intended to represent various forms of digital computers, such as a laptop, a desktop, a workstation, a personal digital assistant, a server, a blade server, a mainframe computer, and other suitable computers. The electronic device can also represent various forms of mobile apparatuses, such as a personal digital assistant, a cellular phone, a smartphone, a wearable device (such as a helmet, glasses, or a watch, etc.), and other similar computing apparatuses. The components shown in this specification, connections and relationships therebetween, and functions thereof are only examples and are not intended to limit the implementation of the present disclosure described and/or claimed in this specification.

As shown in FIG. 7, the electronic device 10 includes at least one processor 11 and a memory in a communication connection to the at least one processor 11, such as a read-only memory (ROM) 12 or a random access memory (RAM) 13. The memory stores a computer program that can be executed by the at least one processor. The processor 11 can perform various appropriate actions and processing based on a computer program stored in the read-only memory (ROM) 12 or a computer program loaded from a storage unit 18 into the random access memory (RAM) 13. In the RAM 13, various programs and data required for operations of the electronic device 10 can also be stored. The processor 11, the ROM 12, and the RAM 13 are connected to each other by using a bus 14. An input/output (I/O) interface 15 is also connected to the bus 14.

A plurality of components of the electronic device 10 are connected to the I/O interface 15, including an input unit 16, such as a keyboard or a mouse; an output unit 17, such as various types of displays and speakers; the storage unit 18, such as a disk or an optical disc; and a communication unit 19, such as a network card, a modem, or a wireless communication transceiver. The communication unit 19 allows the electronic device 10 to exchange information/data with other device through a computer network such as the Internet and/or various telecommunications networks.

The processor 11 can be various general and/or dedicated processing component with processing and computing capabilities. Some examples of the processor 11 include but are not limited to a central processing unit (CPU), a graphics processing unit (GPU), various specialized artificial intelligence (AI) computing chips, various processors running machine learning model algorithms, a digital signal processor (DSP), and any appropriate processor, controller and microcontroller. The processor 11 implements various methods and processes described above, such as the feature screening method.

In some embodiments, the feature screening method can be implemented as a computer program, which is tangibly included in a computer readable storage medium, such as the storage unit 18. In some embodiments, a part or all of the computer program can be loaded and/or installed on the electronic device 10 by using the ROM 12 and/or the communication unit 19. When the computer program is loaded into the RAM 13 and is executed by the processor 11, one or more steps of the feature screening method described above can be implemented. Alternatively, in other embodiments, the processor 11 can be configured to implement the feature screening method by any other appropriate means (for example, by using firmware).

Various implementations of the systems and technologies described above in this specification can be implemented in a digital electronic circuit system, an integrated circuit system, a field programmable gate array (FPGA), an application specific integrated circuit (ASIC), an application specific standard product (ASSP), a system on chip (SOC), a complex programmable logic device (CPLD), computer hardware, firmware, software, and/or combinations thereof. The various implementations can include implementation in one or more computer programs. The one or more computer programs can be executed and/or interpreted on a programmable system including at least one programmable processor. The programmable processor can be a dedicated or universal programmable processor that can receive data and instructions from a storage system, at least one input apparatus, and at least one output apparatus, and transmit the data and the instructions to the storage system, the at least one input device, and the at least one output device.

The computer program for implementing the feature screening method of the present disclosure can be written in any combination of one or more programming languages. These computer programs can be provided to a processor of a general-purpose computer, a special-purpose computer, or other programmable data processing apparatus, so that functions/operations specified in a flowchart and/or a block diagram can be implemented when the computer program is executed by the processor. The computer program can be entirely or partially executed on a machine; as an independent software package, can be partially executed on the machine and partially executed on a remote machine, or is completely executed on the remote machine or a server.

### Embodiment 7

Embodiment 7 of the present disclosure provides a computer readable storage medium. The computer readable storage medium stores computer instructions, and the computer instructions are used to enable a processor to implement a feature screening method, wherein the method includes:
determining a plurality of feature validation subsets based on data features in sample data;
performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
training a machine learning model of a processing target based on the training dataset and the validation dataset corresponding to each feature validation subset; and
determining a target data feature group corresponding to the processing target based on training process data of each machine learning model.

In the context of the present disclosure, the computer readable storage medium may be a tangible medium that includes or stores a computer program for use by or in combination with an instruction execution system, device, or apparatus. The computer readable storage medium may include, but is not limited to, an electronic, a magnetic, an optical, an electromagnetic, an infrared, or a semiconductor system, apparatus, or device, or any suitable combination thereof. Alternatively, the computer readable storage medium can be a machine readable signal medium. More specific examples of the machine readable storage medium may include an electrical connection with one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof.

To provide interactions with a user, the system and the technology described herein can be implemented on an electronic device. The electronic device has a display apparatus (such as a CRT (cathode ray tube) or an LCD (liquid crystal display) monitor) for displaying information to the user, a keyboard, and a pointing apparatus (such as a mouse or a trackball). The keyboard and the pointing device can be used to provide input to the electronic device by the user. Other types of apparatuses can also be used to provide interactions with the user. For example, feedback provided to the user can be any form of sensory feedback (such as visual feedback, auditory feedback, or tactile feedback). Moreover, input from the user can be received in any form (including voice input, speech input, or tactile input).

The system and the technology described herein can be implemented in a computing system that includes a back-end component (for example serving as a data server), or a computing system that includes a middleware component (such as an application server), a computing system that includes a front-end component (such as a user computer with a graphical user interface or a web browser through which the user can interact with implementation of the system and the technology described herein), or a computing system that includes any combination of such back-end component, such middleware component, or such front-end component. The components of the system can be interconnected through digital data communication (such as a communication network) in any form or of any medium. Examples of the communication network include a local area network (LAN), a wide area network (WAN), a blockchain network, and the Internet.

The computing system may include a client and a server. The client and the server are generally far away from each other and usually interact through the communication network. A relationship between the client and the server is generated by using computer programs that are run on a corresponding computer and have a client-server relationship with each other. The server can be a cloud server, which is also referred to as a cloud computing server or a cloud host, and is a host product in a cloud computing service system, to resolve shortcomings of high management difficulty and weak business scalability in traditional physical hosts and VPS services.

It should be understood that various forms of processes shown above can be used for reordering, adding, or deleting steps. For example, the steps described in the present disclosure can be implemented in parallel, in sequence, or in different orders, provided that an expected result of the technical solutions of the present disclosure can be achieved. This is not limit in this specification.

The foregoing specific implementations do not constitute any limitation on the protection scope of the present disclosure. A person skilled in the art should understand that various modifications, combinations, sub combinations, and substitutions can be made based on design requirements and other factors. Any modification, equivalent replacement, improvement, and the like made within the spirit and principles of the present disclosure shall fall within the protection scope of the present disclosure.

1. A feature screening method, comprising:
determining a plurality of feature validation subsets based on data features in sample data;
performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
training a machine learning model of a processing target based on the training dataset and the validation dataset corresponding to each feature validation subset; and
determining a target data feature group corresponding to the processing target based on training process data of each machine learning model.

2. The method according to claim 1, wherein before the determining a plurality of feature validation subsets based on data features in sample data, the method further comprises:
determining association between each data feature in the sample data and the processing target, and screening out a candidate data feature based on the association between the data feature and the processing target; and
correspondingly, the determining a plurality of feature validation subsets based on data features in sample data comprises: determining a plurality of feature validation subsets in the candidate data feature.

3. The method according to claim 1, wherein the determining a plurality of feature validation subsets based on data features in sample data comprises:
determining a plurality of feature validation subsets in the data feature in the sample data or in the candidate data feature based on a quantity of features in the feature validation subsets.

4. The method according to claim 1, wherein the performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning comprises:
partitioning at least one group of sample data of a same individual to one individual group, to obtain individual sample groups corresponding to different individuals; and performing the cross-validation partitioning on the plurality of individual sample groups based on at least one preset cross validation rule, to determine the training dataset and the validation dataset that are obtained through partitioning;
and/or
the determining a target data feature group corresponding to the processing target based on training process data of each machine learning model comprises:
for any machine learning model, respectively determining a training indicator and a test indicator based on training data and validation data in the training process data of the machine learning model; sequencing and screening various machine learning models based on the training indicator and the test indicator of each machine learning model; and determining a feature validation subset corresponding to a screened machine learning model as the target data feature group of the processing target,
wherein the training indicator and the test indicator respectively comprise a root-mean-square error and a goodness of fit.

5. The method according to claim 1, wherein after determining the target data feature group, the method further comprises:
for any target data feature, drawing a data distribution map of the target data feature based on sample data corresponding to the target data feature; and
validating the target data feature based on the data distribution map of the target data feature.

6. The method according to claim 5, wherein the drawing a data distribution map of the target data feature based on sample data corresponding to the target data feature comprises:
determining a data type of the target data feature; and drawing a data distribution map whose type corresponds to the data type based on the sample data corresponding to the target data feature;
and/or
the validating the target data feature based on the data distribution map of the target data feature comprises: in response to that the data distribution map of the target data feature does not conform to a distribution rule, removing the target data feature, or removing a target data feature group to which the target data feature belongs.

7. The method according to claim 6, wherein the determining a data type of the target data feature comprises:
performing deduplication on data values of the target data feature to obtain deduplicated data values; in response to that each deduplicated data value is an integer and a quantity of data values is less than or equal to a preset threshold, determining that the data type of the target data feature is a sub type; and in response to that each deduplicated data value is not an integer or the quantity of the data values is larger than or equal to the preset threshold, determining that the data type of the target data feature is a numerical type;
and/or
the drawing a data distribution map corresponding to the data type based on the sample data corresponding to the target data feature comprises:
if the data type of the target data feature is the sub type, drawing, based on the sample data corresponding to the target data feature, a horizontal bar chart of the target data feature, and a box chart of the target data feature and the processing target; if the data type of the target data feature is the numerical type, drawing, based on the sample data corresponding to the target data feature, a histogram of the target data feature, and a scatter regression plot of the target data feature and the processing target.

8. (canceled)

9. An electronic device, wherein the electronic device comprises:
at least one processor; and
a memory in a communication connection with the at least one processor,
wherein the memory stores a computer program that can be executed by the at least one processor, and the computer program is executed by the at least one processor to enable the at least one processor to implement a feature screening method,
wherein the feature screening method comprises:
determining a plurality of feature validation subsets based on data features in sample data;
performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
training a machine learning model of a processing target based on the training dataset and the validation dataset corresponding to each feature validation subset; and
determining a target data feature group corresponding to the processing target based on training process data of each machine learning model.

10. A tangible computer readable storage medium, wherein the computer readable storage medium stores computer instructions, and the computer instructions are used to enable a processor to implement a feature screening method,
wherein the feature screening method comprises:
determining a plurality of feature validation subsets based on data features in sample data;
performing, based on an individual to which the sample data belongs, individual group partitioning on the sample data to obtain individual sample groups corresponding to different individuals, and performing cross-validation partitioning based on a plurality of individual sample groups, to determine a training dataset and a validation dataset that are obtained through partitioning;
training a machine learning model of a processing target based on the training dataset and the validation dataset corresponding to each feature validation subset; and
determining a target data feature group corresponding to the processing target based on training process data of each machine learning model.
